(19) United States
(12) Patent Application Publication
(10) Pub. No.: US 2020/0054267 A1
(43) Pub. Date: Feb. 20, 2020

(54) METHOD, SYSTEM AND APPARATUS FOR DETECTION OF NEURO ATTACKS

(71) Applicant: S SQUARE DETECT MEDICAL DEVICES, Andover, MA (US)

(72) Inventor: Govindarajan T. SRINIVASAN, Andover, MA (US)

(21) Appl. No.: 16/663,136

(22) Filed: Oct. 24, 2019

Related U.S. Application Data
(63) Continuation-in-part of application No. 15/614,614, filed on Jun. 6, 2017.
(60) Provisional application No. 62/346,172, filed on Jun. 6, 2016.

(51) Int. Cl. A61B 5/00; A61B 5/026

(52) U.S. Cl. CPC A61B 5/4064; A61B 5/0075; A61B 5/6814; A61B 5/7282; A61B 5/7225; A61B 5/0261

(57) ABSTRACT

A portable neuro attack monitoring device is described. The neuro attack monitoring device combined with Coherent Hemodynamic Spectroscopy CHS algorithm offers a unique opportunity to directly resolve blood flow velocity measurements and for the first time apply NIRS+CHS technique for the detection of ischemic strokes and TIA. The device comprises a central hub configured for placement on a central part of a patient's head and a plurality of spokes connected to the central hub and configured for placement on the patient's head over a specific portion of the patient's brain. Each spoke can comprise one or more pairs of light emitting sources and at least one light detector, and the light emitting sources can be configured to inject light into the patient's head, at two or more different wavelengths, over a predetermined period of time.

### RELATED APPLICATIONS

This application claims priority to and is a Continuation-in-Part of U.S. application Ser. No. 15/614,614, filed on Jun. 6, 2017, which claims priority to U.S. Provisional Application No. 62/346,172, filed on Jun. 6, 2016. The entire teachings of the earlier applications are incorporated by reference herein.

### TECHNICAL FIELD

The present disclosure relates generally to neuro attack monitoring and, more particularly to methods, apparatus, and systems for detection of ischemic attack events.

### BACKGROUND

According to the World Health Organization (WHO), nearly fifteen million people can die from complications associated with having a stroke annually. For example, in the United States, nearly seven million people are thought to have experienced a stroke, and nearly 150,000 deaths are caused by complications associated with having a stroke every year.

Ischemic attack events, such as Transient Ischemic Attack (TIA) events can be a precursor or warning indicator for an impending and disabling ischemic stroke. Although, every year, nearly 500,000 patients are estimated to have experienced a TIA event, less than 40% of such patients are believed to have received medical help. This can be due to the fact that the symptoms of a typical TIA event often lasts for less than fifteen minutes, allowing the patient to feel completely normal after experiencing a TIA event and, thereby, providing the patient with false reassurance.

However, it is estimated that approximately 15% of the patients who experience a TIA event will have an ischemic stroke within about three months of the TIA event. An ischemic stroke can result in the patient's death or leave the patient with permanent long-term disability. In addition to personal and emotional burdens on the patients affected by strokes, the long-term disability caused by the stroke can result in an economic burden on the society.

### SUMMARY

The present disclosure relates to methods, apparatus, and systems for detection and prevention of ischemic attack events, such as Transient Ischemic Attack (TIA) events.

In one aspect, a portable monitoring device, which is herein also referred to a Neuro Attack Prevention (NAP) device, is disclosed. The NAP device combined with Coherent Hemodynamic Spectroscopy (CHS) computational methods can offer a unique opportunity to directly resolve blood flow velocity measurements and for the first time apply near-infrared spectroscopy combined with CHS techniques for the detection of ischemic strokes and TIA.

In some embodiments, the device can include a central hub configured for placement on a patient's head, for example, on a central part of a patient's head, and a plurality of spokes connected to the central hub and configured for placement on the patient's head, and in particular over specific portions of the patient's brain. In some embodiments, each spoke can comprise one or more pairs of light emitting sources and at least one light detector, and the light emitting sources can be configured to inject light into the patient's head, at two or more different wavelengths, over a predetermined period of time.

In one aspect, a neuro attack monitoring device is disclosed, which comprises a central hub configured for placement on a central part of a patient's head; and a plurality of probes connected to the central hub and configured for placement on the patient's head over a specific portion of the patient's brain, at least one of said probes comprising one or more pairs of light emitting sources and at least one light detector; wherein the light emitting sources are configured to inject light into the patient's head, at two or more different wavelengths, over a predetermined period of time.

In some embodiments, at least one of the probes comprises two portions that are hingedly-coupled to one another via a swivel hinge. In some such embodiments, the swivel hinge is configured to allow adjusting an angle between said probes in a range of about 15° to about 30° degrees. In some such embodiments, each of the hingedly-coupled probe portions comprises a detector. Further, in some such embodiments, in addition to the detector, each of the hingedly-coupled probe portions can include a pair of emitters.

In some embodiments, each probe comprises a spoke extending from the central hub to a pod (a head portion). In some such embodiments, at least one of the pods associated with at least one of the probes is configured to house a pair of light emitting sources and at least one photodetector. By way of example, the photodetector can be a photodiode, such as an avalanche photodiode.

In some embodiments, at least one of the pairs of emitters generates radiation at a wavelength in a range of about 650 nm to about 710 nm and another one of the pairs of emitters generates radiation at a wavelength in a range of about 800 nm to about 830 nm. In some embodiments of the device, the source wavelengths in the range of 690 nm-830 nm can be switched between different wavelengths or pulsed using the same emitter using a time delay circuitry in the hub.

In some embodiments, the device can further include a controller for controlling the operation of the light emitting sources and the light detector. In some such embodiments, the controller is positioned in the central hub.

In a related aspect, a system for neuro attack monitoring is disclosed, which comprises a monitoring device and a computing device that is in communication with the monitoring device. The monitoring device can include a central hub configured for placement on a central part of a patient's head; and a plurality of probes connected to the central hub and configured for placement on the patient's head over a specific portion of the patient's brain, at least one of said probes comprising one or more pairs of light emitting sources and at least one light detector; wherein the light emitting sources are configured to inject light into the patient's head, at two or more different wavelengths, over a predetermined period of time. The computing device can receive data generated by the light detector and operate on that data to determine the onset of an ischemic event.

In some such embodiments, the computing device can employ Coherent Hemodynamic Spectroscopy (CHS), a mathematical computational method, to extract parameters of interest, such as cerebral blood transit times, Cerebral Blood Flow velocity (CBFᵥ), Cerebral Blood Volume (CBV) and cerebral autoregulation for the obtained data.

In other examples, any of the aspects above, or any system, method, apparatus described herein can include one or more of the following features.

Other aspects and advantages of the invention can become apparent from the following drawings and description, all of which illustrate the principles of the invention, by way of example only.

### BRIEF DESCRIPTION OF THE DRAWINGS

Features and advantages of the invention described herein, together with further advantages, may be better understood by referring to the following description taken in conjunction with the accompanying drawings. The drawings are not necessarily to scale, emphasis instead is generally placed upon illustrating the principles of the invention.

FIG. 1A illustrates an example of a neuro attack monitoring system according to some embodiments disclosed herein.

FIG. 1B schematically illustrates various components of the neuro attack monitoring system shown in FIG. 1A.

FIG. 2A illustrates an example of a neuro attack monitoring device according to some embodiments disclosed herein.

FIG. 2B schematically illustrates a pair of radiation emitters and a detector employed in a probe of a neuro attack monitoring system according to some embodiments.

FIG. 3 schematically illustrates a probe employed in a neuro attack monitoring system according to an embodiment, which includes two portions that are hingedly coupled to one another.

FIGS. 4A, 4B, and 4C schematically depict an example of positioning of the probes of a neuro attack monitoring system according to an embodiment on a subject's skull.

FIG. 4D schematically depicts peripheral devices, such as a pressure cuff, that can be coupled to a subject.

FIG. 5 schematically depicts one example of the implementation of a hub of a neuro attack monitoring device according to an embodiment.

FIG. 6 is a block diagram depicting various components of a neuro attack monitoring system according to an embodiment.

FIG. 7 is another block diagram depicts various components of a neuro attack monitoring system according to an embodiment.

FIG. 8 schematically depicts the coupling of a peripheral device to a subject.

FIG. 9 is a flow chart depicting various steps for operating a NAP device according to an embodiment of the present teachings.

FIG. 10 shows the flow of data in a system according to the present teachings.

FIG. 11 shows typical spectrum of an IR and red diode emitter.

FIG. 12 presents extinction coefficients for oxy and deoxy hemoglobin.

FIG. 13 shows calculated Differential Path Factor (DPF) of an adult brain as a function of wavelength. The DPF is a function of the light wavelength and other biological factors, such as patient's age and tissue composition, and

FIG. 14 shows the data with the signal values of the de-oxygenated hemoglobin (Hb) concentration and oxygenated hemoglobin (HbO2) concentration as modeled by the NAP physical model.

### DETAILED DESCRIPTION

The present disclosure relates to methods, apparatus, and systems for detection and prevention of ischemic attack events.

The term “ischemia,” as used herein, generally refers to a restriction in blood supply to tissues that can cause a shortage of oxygen needed for cellular metabolism. An ischemic stroke can generally occur as a result of an obstruction within a blood vessel supplying blood to the brain. A Transient Ischemic Attack (TIA) generally refers to a temporary blockage of blood flow to the brain. A TIA does not usually cause permanent damage and can be potentially ignored by the patient since the symptoms are often temporary and generally last less than five minutes. Once a TIA is over, the particular blockage causing the TIA usually causes no permanent injury to the brain. The blockage is often short-term or temporary during a TIA and the blockage can often dissolve on its own or get dislodged so that the symptoms disappear.

An ischemic attack event, such as a Transient Ischemic Attack event (TIA), can, however, be an important warning sign because it can signal a problem that can lead to an impending and disabling ischemic stroke, subsequent strokes, or even death. Therefore, detection and identification of such events can potentially assist in prevention of possible later strokes.

However, most existing techniques for detection of TIA events are inaccurate, inefficient, subjective, and expensive. Presently, diagnostic assessment schemes for ischemic stroke and TIA diagnosis are mainly dominated by Magnetic Resonance Imaging (MRI) with diffusion weighted imaging and perfusion weighted imaging. Although Magnetic Resonance Imaging (MRI) can potentially detect such TIA events, MRI-based techniques are not always part of the standard-of-care in acute settings such as Emergency Rooms (ER). Further, MRI-based techniques are often costly and require access to non-portable imaging machines. Therefore, such techniques cannot easily be used to form an affordable and/or portable diagnostic tool.

Other detection schemes, such as Transcranial Doppler Devices (TCD), impose similar difficulties. For example, although TCD-based schemes can allow for direct measurements of blood flow velocity, they cannot be used to determine the state of the blood supplied to the tissue. Further, the capability of TCD-based schemes for monitoring the blood flow velocity can be limited by how ultrasound beams penetrate the specific patient's temporal bone anatomy. Additionally, the blood flow velocity measurements provided by TCD generally reflect macro-vascular changes, thereby reducing its specificity as a valid diagnostic tool for small vessel or branch vessel occlusions that usually cause ischemic strokes or TIA events.

The present disclosure addresses the need for TIA detection capability by providing methods, systems, and corresponding devices that allow for portable and compact diagnostic and prediction of ischemic attacks both inside and outside of the hospital environment (e.g., at home and in ambulatory care).

As discussed in more detail below, the present disclosure provides a system for detection of stroke and/or TIA soon after its onset, which includes a device configured for placement on a patient's head for illuminating a specific region of the patient's brain with radiation (e.g., infrared radiation) and detecting at least a portion of the radiation after its transmission through at least a portion of that brain region. The data can be collected and analyzed using Coherent Hemodynamic Spectroscopy (CHS), as discussed in more detail below.

CHS is a technique that relies on systemic Arterial Blood Pressure (ABP) oscillations (which may occur spontaneously or may be safely induced by timed inflation and deflation of pneumatic cuffs on a subject's arm or thigh) and Near Infra-Red Spectroscopy (NIRS) measurements of the resulting, coherent cerebral hemodynamic oscillations. Such hemodynamics is then analyzed with a mathematical model to translate them into quantitative measures of blood capillary transit times, cerebral blood flow, and cerebral autoregulation. The mathematical model focuses on the time spent by the red blood cells traveling through the capillary compartment, and the rate constant for oxygen release to the surrounding tissue. The CHS model treats the cerebral microcirculation as a Linear Time Invariant (LTI) system, for which the inputs are the cerebral blood volume, blood flow, and oxygen consumption, and the outputs are the brain concentrations of oxyhemoglobin (O), deoxyhemoglobin (D), and total hemoglobin (T).

CHS has been employed to measure cerebral hemodynamics at the microcirculatory level in patients undergoing hemodialysis. For example, some such studies have used cyclic inflation and deflation of a pneumatic cuff placed around a subject's thigh at multiple frequencies, e.g., frequencies in a range of 0.03 to 0.17 Hz, to generate CHS spectra and to obtain a set of physiological parameters that include the blood transit times in the cerebral microcirculation, the cut-off frequency of cerebral autoregulation, and blood volume ratio. Some such studies showed that hemodialysis resulted in a mild reduction in Cerebral Blood Flow velocity (CBFv). Such studies are relevant to stroke because, in hemodialysis, blood gets filtered outside the body with increased viscosity. This mimics stroke as an increase in viscosity causes reduction in CBFv and Cerebral Blood Volume (CBV). Further, CHS has been used to measure brain perfusion and cerebral autoregulation in patients in neurological care units.

The present disclosure is based at least in part on the recognition that measurements of changes in CBF in real-time and non-invasively can be utilized to diagnose acute ischemic stroke and/or TIA soon after its onset.

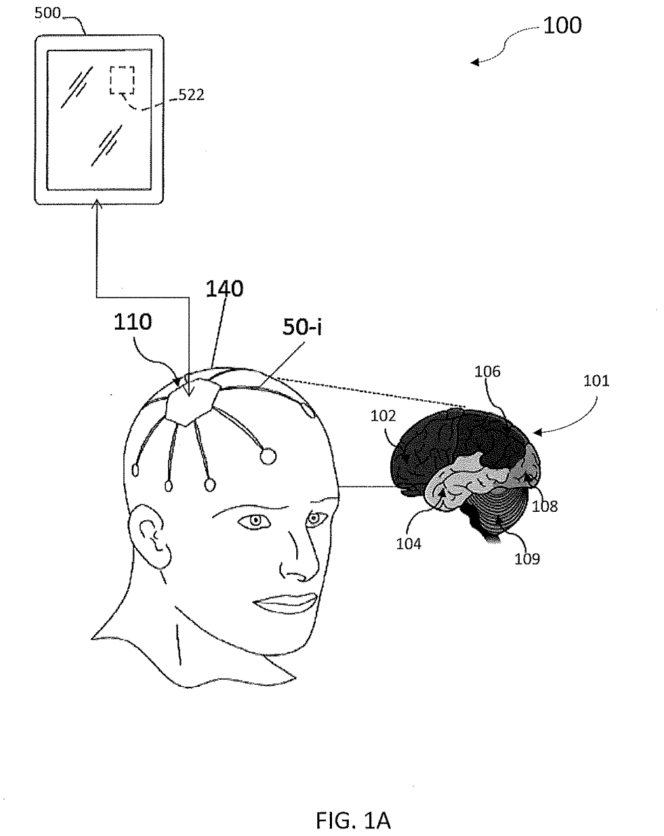

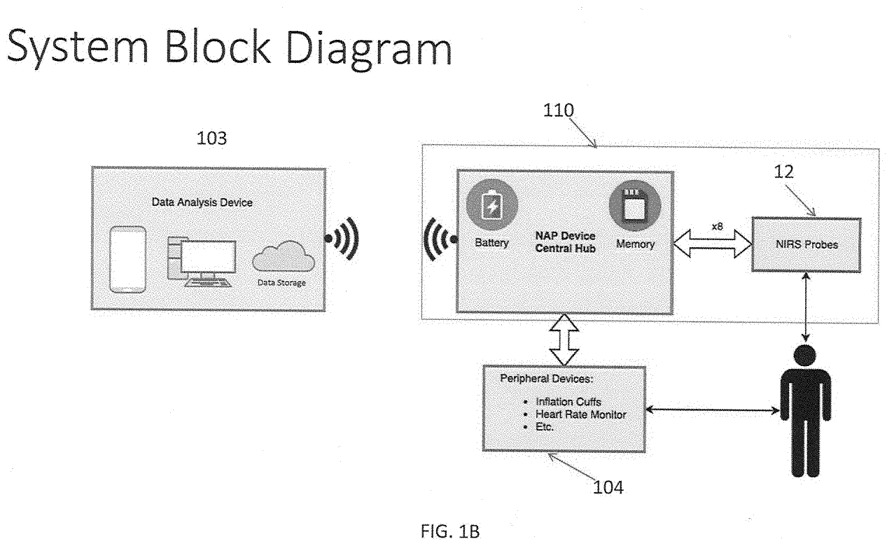

With reference to FIGS. 1A and 1B, an example of a neuro attack monitoring system 100 according to some embodiments, includes a neuro attack monitoring device (herein also referred to as a Neuro Attack Prevention (NAP) device) 110 that is configured for placement on a patient's head 140. The NAP device 110 can generally be placed at any suitable location on the patient's head 140. For example, as shown in FIG. 1A, the NAP device 110 can be placed on or near the center of the patient's head 140. Additionally or alternatively, the NAP device 110 can be configured for placement on side or corner of the patient's head 140. For example, the NAP device 110 can be placed such that it is centered on one side of the patient's head 140.

The NAP device 110 can include a plurality of probes 12, each of which is configured to emit infrared radiation to illuminate at least one region of the subject's brain and to detect radiation transmitted through one or more illuminated regions of the subject's brain in response to such illumination of the subject's brain, as discussed in more detail below. Further, the neuro attack monitoring system 100 can include peripheral devices 104 such as one or more inflation cuffs (e.g., for inducing hemodynamic oscillations), a heart monitor, etc. The neuro attack monitoring system 100 is in communication, e.g., via a wireless, Bluetooth, Zigbee or Near Field Communication (NFC) protocol, with a data analysis device and/or cloud servers 103 that can receive the signals generated by the probes 12.

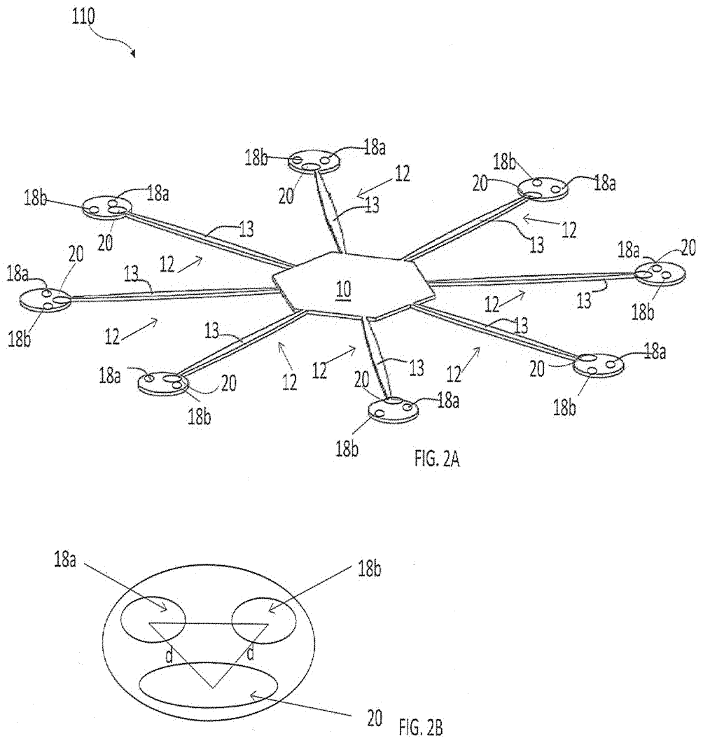

As shown in FIG. 2A, in this embodiment, the device 110 comprises eight probes 12 emanating from a centralized hub 10. Each probe 12 includes a spoke 13 that extends from the hub 10 to head portion (herein also referred to as pod) 15, in which a coupled pair of emitters 18a/18b (collectively referred to as light emitters 18 or light sources 18), e.g., Light Emitting Diodes (LEDs) and a detector 20, e.g., a photodiode, are positioned. In some embodiments, the light emitters can be in the form of a dual band laser or a light emitting diode (LED), for example, with a bandwidth range of 10-20 nm. In this embodiment, the light emitters and detector are positioned at a distance in a range of about 2 cm to about 3 cm relative to one another. By way of example, as shown in FIG. 2B, in some embodiments, the detector can be positioned below the light emitters along a putative line that bisects another putative line connecting the two light emitters at a distance d, which is the distance from the center of the source to the center of the detector, and which can be in a range of about 2 cm to about 3 cm.

In this embodiment, the light emitters 18 can be activated to generate light at two different wavelengths, for example, at wavelengths of 690 nm and 830 nm. For example, each of the light emitters 18 can be a solid state laser that can provide different radiation wavelengths in response to different pulsing frequencies. By way of example, in some embodiments, a light emitting source can be configured with a single IR laser with a fiber beam splitter, which can feed all sources through a plurality of fiber optic cables. In some embodiments of the device, the source wavelengths of the above IR laser or LED that is in the range of 690 nm-830 nm can be switched between two or more wavelengths (e.g., via pulsing the same emitter using a time delay in the hub).

In some embodiments, the light detector can be based on an avalanche photodiode (APD) technology. For example, the light detector can be a standard silicon APD manufactured and sold by Hamamatsu Inc. Typical performance specifications for such low cost device include: a spectral sensitivity range of 400 to 1000 nm, with a peak sensitivity at 800 nm. In addition, such detectors have high sensitivity and low noise, while operating at relatively low break down voltage, for example approximately 150 V.

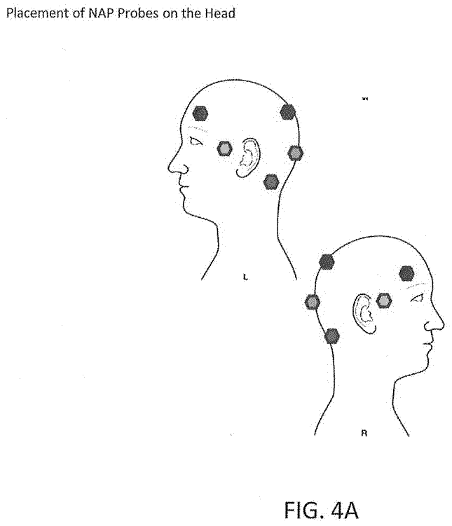

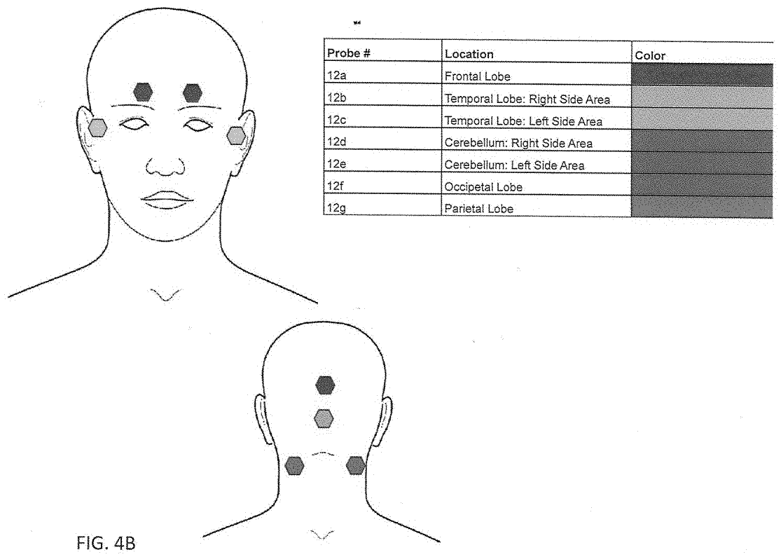

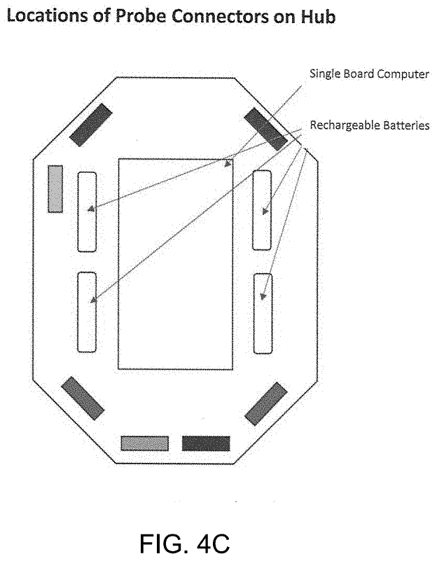

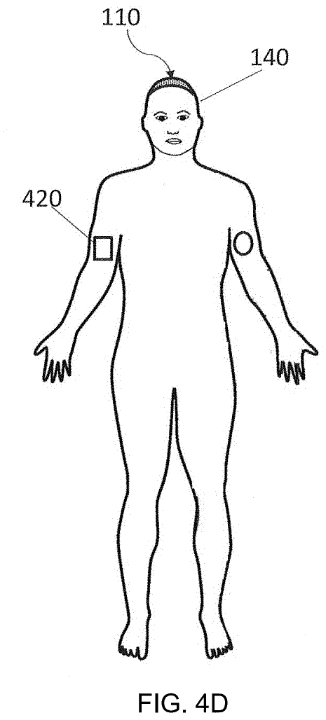

As shown in FIGS. 4A, 4B, and 4C, each probe 12, along with its associated light emitters and detector(s), can be configured such that the radiation emitted from the light emitters associated therewith can target a specific portion of the patient's brain 101. For example, as shown schematically in FIGS. 4A and 4B, the NAP device 110 can be configured such that at least one probe 12a can be positioned on a patient's skull so as to target the frontal lobe of the brain, at least one probe 12b can be positioned on a patient's skull so as to target the right side temporal lobe, at least one probe 12c can be positioned on a patient's skull so as to target the left side temporal lobe, at least one probe 12d can be positioned on a patient's skull so as to target the right side of the area close to the patient's cerebellum, at least one probe 12e can be positioned on a patient's skull so as to target the left side of the area close to the patient's cerebellum, at least one probe 12f can be positioned on the patient's skull so as to target the patient's occipital lobe, and at least one probe 12g can be positioned on the patient's skull so as to target the patient's parietal lobe.

In this embodiment, the light emitted by the probes can collectively cover an area of about 48 cm² of the brain (˜6 cm² per probe).

In some embodiments, at least one of the probes is placed on the subject's forehead, which covers the frontal cortex. As the forehead has no hair, it would not interfere with a detected signal.

The NAP device 110 can be manufactured using any suitable material available in the art, such as ceramic, plastics, or carbon fiber. The hub 10 can contain data acquisition electronics, which in some embodiments can be incorporated in layers of Kapton or FR-4 (grade designation for industry standard composite material of certain type) and Ball Grid Arrays (BGA)s and other electronic components. The NAP device can be configured such that it fits the footprint of a human head.

The hub 10 can house various components. For example, the hub 10 can include any suitable hardware, electronics, data storage unit, and/or software protocols for operating the NAP device 110. The hub 10 can further include suitable communications hardware or software protocol for operation of the NAP device 110. For example, the hub 10 can include the required software and hardware components for either or a combination of wireless, Bluetooth, Zigbee and/or NFC communication. In this embodiment, the hub 10 can house the data acquisition and communication electronics.

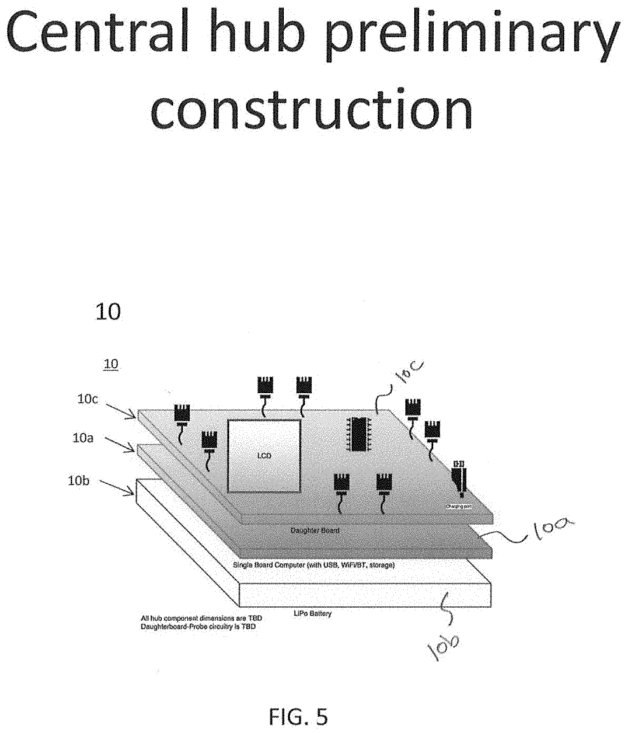

By way of example, FIG. 5 schematically depicts an example of implementation of the hub 10, which includes a layer 10a in which the requisite electronics (such as control electronics) are disposed for operating the NAP device 110. By way of example, the layer 10a can include a PCB on which at least one processor, a plurality of memory modules, and communications buses as well as communications modules for communicating with the data analysis device 103 (See, FIG. 1B) are disposed. By way of example, instructions for operating the NAP device can be stored in at least one of the memory modules and can be accessed by the processor during operation for operating and controlling the NAP device. In this embodiment, the NAP device further includes a layer 10b in which a battery (such as an LiPo battery) is disposed. The NAP device can further include a layer 10c in which an LCD screen and various connectors for connecting the NAP device to external devices, such as charging units, are disposed.

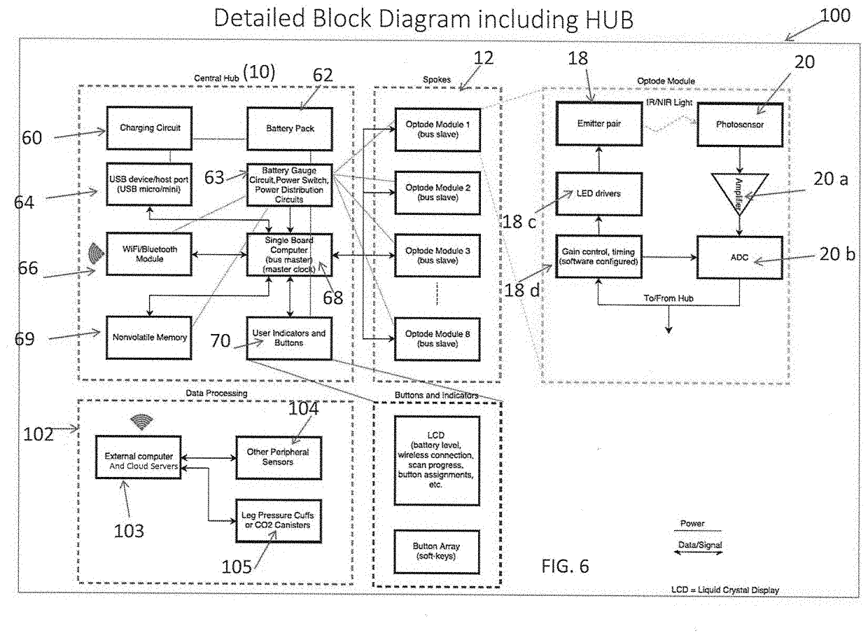

By way of further illustration, with reference to FIG. 6, the hub 10 can include a charging circuit 60 in communication with a battery pack 62 for charging the battery, which is in turn in communication with a battery gauge/power switch/power distribution circuit 63. The hub 10 can further include a USB device 64, and a WiFi/Bluetooth/Zigbee/NFC module 66 for communicating with external devices and/or cloud servers, such as a data processing unit module 102.

The hub 10 can further include a controller 68, which comprises a single board computer and non-volatile memory for operating the NAP device 110 including controlling the probes 12, such as their light sources. For example, the controller 68 can provide alternate activation of the light sources, e.g., by changing the frequency of activation pulses applied to the light sources so as to irradiate the selected portions of the patient's brain with light at wavelengths of 690 nm and 830 nm in different time intervals introduced by a time delay. The hub 10 can further include a user graphical interface, including an LCD and a button array (e.g., a plurality of soft keys) to present information to a user and allow the user to operate the device as well as non-volatile memory 69.

As shown in FIG. 6, in this embodiment, each probe 12 includes a pair of light emitters 18 (e.g., LED emitters) that are configured to emit radiation at different wavelengths, and a photodetector 20 (herein also referred to as photosensor 20) that detects the radiation transmitted through an illuminated region of a subject's brain 101. Each probe 12 can further include LED drivers 18c as well as gain control and timing software 18d for controlling the emitters. The photosensor (which can be, for example, an avalanche photodiode), can generate signals in response to the detection of radiation. An amplifier 20a can amplify the signal(s) generated by the photosensor and an analog-to-digital converter (ADC) 20b can digitize the signals. The digitized signals can be transmitted to the hub 10, which can in turn transmit the digitized signals to data processing unit 102 for processing in accordance with the present teachings. The data processing unit 102 can include a computer and/or external cloud servers 103, which can be in communication with peripheral devices 104, such as pressure inflation cuffs, heart rate monitor etc. and a CO₂ canister and thigh/leg cuffs 105.

As noted above, in this embodiment, each probe includes a detector that can be used to capture at least a portion of the light that has been transmitted through the irradiated portion of the subject's brain. In some embodiments, the detector of each probe is used to detect the radiation associated with the emitter pair of that probe, which can be configured to emit radiation at two wavelengths in a time-multiplexed manner. The detected light is attenuated, e.g., due to scattering and/or absorption, relative to the light irradiating the brain portion at these two time-multiplexed wavelengths.

In many embodiments, the multiplexing frequency (i.e., the frequency at which the light source is alternatively activated) is selected to be equal or greater than about 3 Hz, e.g., in a range of about 3 Hz to about 10 Hz, consistent with the frequency spectrum of the spontaneous hemodynamic oscillations in the brain where the highest component is due to arterial pulsation (˜1-1.5 Hz). In some embodiments, the power applied to the scalp can be, for example, between about 0.15 and 0.2 Watts/cm². In general, the power density at the scalp can be selected to maximize the signal-to-noise ratio while ensuring that the subject will not experience any burning sensation. By way of example, the signal-to-noise ratio can be at least about 1000.

Referring again to FIG. 2A, in this embodiment, the hub 10 is hexagonal in shape, though in other embodiments it can have other shapes, such as rectangular. The spokes 13 can be coupled to the hub 10 using any suitable technique available in the art. In this embodiment, the hub, the spokes, and the housing for the light sources and the detector can be integrally formed using a flexible material, e.g., Kapton or FR-4. In other embodiments, the hub, the spokes and the housing for the light sources and the detector can be formed as separate components and subsequently assembled using known techniques in the art.

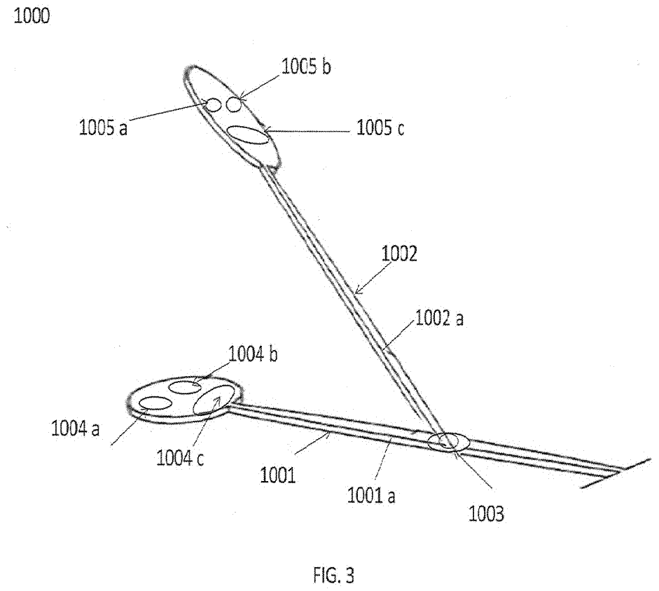

As shown in FIG. 3, in some embodiments, at least one of the probes 1000 includes two portions 1001/1002 that are hingedly coupled to one another via a swivel hinge 1003. More specifically, the spoke 1001a associated with the probe portion 1001 is coupled to the spoke 1002a associated with the probe portion 1002 via the mechanical hinge 1003, which allows the two spokes to swivel relative to one another. In this embodiment, the probe portion 1001 can include a pair of emitters 1004a/1004b and a photodetector 1004c, and the probe portion 1002 includes a pair of emitters 1005a/1005b and a photodetector 1005c. In other embodiments, one of the probe portions can include only a pair of emitters and the other probe portion can include only a photodetector.

The mechanical hinge 1003 forms a swivel design and is located in the middle of the spoke 1001a that emanates from the hub. The hinge 1003 allows the swivel of the spoke 1002a relative to the spoke 1001a, thus adjusting the distance between the two detectors associated with these two probe portions. In some embodiments, the mechanical hinge 1003 allows adjusting the angle between the spoke 1001a and 1002, for example, in a range of about 15 to about 30 degrees. In such a configuration (i.e., with the spokes 1001a and 1002 angled relative to one another), the NAP device 110 obtains multiplexed data from the two different detectors associated with the spokes 1001a and 1002, which are separated by an adjustable distance. In particular, in this embodiment, the detector d1 is at a distance of 3 cm from the source and the detector d2 is at a distance multiplied by the cosine of the angle formed between the source-detector pair. This allows the NAP data handling software to obtain the signal that has an improved signal to noise ratio of 1/(distance) squared. The swivel design of the flexible spoke enables a dynamic (real time) acquisition of synchronous and asynchronous time-multiplexed optical signals.

The NAP device can include any suitable number of probes 12, and the probes can be positioned at any suitable orientation with respect to the hub 10. For example, in one embodiment, the NAP device 10 can include at least eight probes emanating from a centralized hub 10.

Generally, the light emitting sources associated with the probes 12 can be configured to operate in any manner known in the art. For example, the light emitting sources can be configured such that they emit light beams simultaneously. Alternatively, the light emitting sources can be configured to emit light beams in a temporal sequence.

The detectors employed in a device according to the present teachings, such as the detectors 20/1004c/1005c, can also be configured to operate in any manner known in the art. For example, each detector can be configured to receive the light emitted by the light-emitting source associated therewith or to receive light emitted from another light emitting source (after transmission through a region of the subject's brain). For example, in certain embodiments, a detector employed in a device according to the present teachings can be configured to receive the light emitted by light source(s) associated with one or more neighboring or adjacent probes.

The NAP device 110 can be configured such that, upon placement on the patient's head 140, the device 110 serves as a multi-array sensor that can provide a comprehensive hemodynamic assessment of the patient's brain 101. By using the detector associated with each probe to perform multi-distance measurements of the data emitted by the plurality of sources disposed at different distances relative to that detector, the NAP device 110 can discern depth information, e.g., in a range of about 1.5 cm-2 cm, of skull and up to 3 cm of tissue. For example, the detector associated with a probe can detect radiation emitted by two light sources associated with the adjacent probes after the transmission of the light through a region of the subject's brain. In some embodiments having two probe portions that are hingedly coupled (See, e.g., FIG. 3 above), one probe portion can swivel around the other such that the spokes associated with the two probe portions form an angle in a range of about 15 to about 30 degrees relative to one another. Such a hinge design allows dynamic determination of an optimal separation between the source associated with one probe portion and a detector associated with a different probe portion so as to obtain the desired depth information.

Further, in some embodiments, the multiplexing of the sources and the detectors (e.g., by using the same detector to detect radiation from multiple sources) can allow removing the more superficial hemodynamics contributions, thereby accentuating the cerebral hemodynamic oscillations.

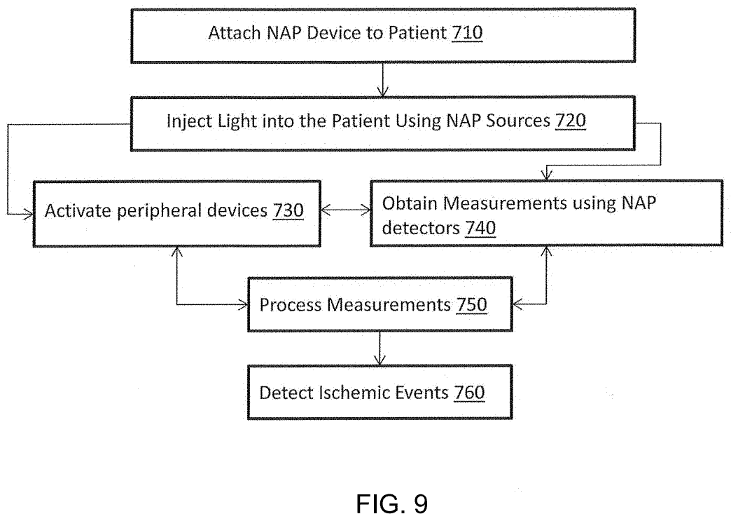

FIG. 9 is a high-level flow diagram of the steps of a procedure that can be employed in one embodiment for detecting an ischemic event using the NAP device 110 according to some embodiments disclosed herein. The device can be operated by being attached to the patient at any suitable position on the patient's head (box 710). The raw data can be acquired by the NAP device using the NAP device (box 720), then sent to NAP data handling and pre-processing algorithm that employs, in some embodiments, a higher-order polynomial interpolation (e.g., 3rd order or higher) and a low pass filter (box 740). This processed data can then be analyzed for oxygenated and de-oxygenated hemoglobin concentration using standard Near Infra-Red Spectroscopy technique. In addition, using these concentration levels, direct measurements of the brain parameters, such as cerebral blood flow, cerebral autoregulation and/or cerebral blood volume, all clinically relevant parameters in detecting ischemic events in the brain can be made (box 750) using any suitable advanced spectroscopy method, one such technique is Coherent Hemodynamic Spectroscopy as discussed in more detail herein and also as outlined in International Publication No. WO2014099124, the entire teachings of which is incorporated by herein by reference in its entirety.

Once attached to the patient, the device can be powered on. The device can be powered on using any suitable available technique, for example directly (e.g., pressing a button on the device) or indirectly (e.g., activating the device using a computing device that may be directly (e.g., via a physical link) or indirectly (e.g., via a wireless link) connected to the NAP device). The NAP device can be used to inject light beams (e.g., infrared beams) into the patient's skull (box 720). The light beams can be injected into the patient's skull at any suitable wavelength/frequency as described above. The computing device and its corresponding software can control the characteristics of the light beams (frequency/wavelength, intensity, modulation, duration, etc.). Peripheral devices can be activated (box 730) to operate in a manner disclosed herein, e.g., to induce the hemodynamic oscillations. The emitted light beams penetrate the patient's head. At least a portion of the light penetrating a region of the patient's brain can exit the skull and be detected (box 740). The light detected in response to the illumination of the patient head can then be analyzed (box 750) using an analysis method known as NIRS-CHS, as disclosed herein and also described in the aforementioned International Publication No. WO 2014099124, to extract the relevant physiological parameters. A NAP classification algorithm can be then used (box 760) to correlate the extracted physiological parameters to the occurrence of ischemic, transient ischemic and/or stroke events in the brain. In one embodiment, a classification algorithm is defined as the threshold the NAP software uses to ascertain the extent of Cerebral Blood Flow velocity (CBFᵥ) present in an adult human brain. A typical CBFᵥ value of 50 mL/100 g/min presents a baseline for a normal adult human brain. In conjunction with the auto-regulation parameter value, the NAP will detect any drop in CBFᵥ greater than 30 mL/100 g/min and classify it as a full ischemic stroke. In conjunction with the auto-regulation parameter value, the NAP will detect any drop in CBFᵥ in the range of 10-15 mL/100 g/min and classify it as a transient ischemic event and/or attack.

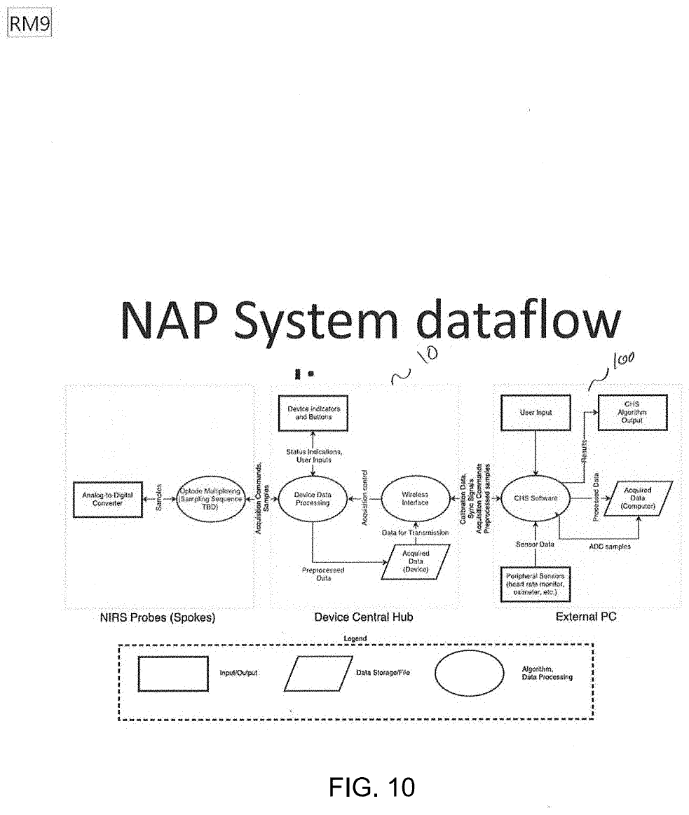

By way of further illustration, FIG. 10 shows the flow of data within a neuro attack monitoring system 100 according to the present teachings. In particular, data obtained by a probe detector (e.g., frequency multiplexed data) can be digitized (via an ADC) and transmitted to the hub 10 which can provide preprocessing of the data (such as a low pass filter or a moving average filter or a box-car filter), and the preprocessed data can be transmitted to a data processing unit (e.g., an external computer 103). In one embodiment, the NAP pre-processing step employs a high order polynomial interpolation followed by the application of a low pass filter. In this embodiment, a copy of the preprocessed data can be stored on the hub 10. The data processing unit can operate on the pre-processed data to extract parameters, e.g., CBFv. More specifically, in this embodiment, the processing unit can include a CHS analysis unit (software) that can receive the preprocessed data as well as data generated by one or more sensors to determine parameters of interest. The processing unit can store the input data as well as the result of the CHS analysis, and can further present the result of the operation of the CHS method on the data to a user. Although in this embodiment, the processing unit is depicted as an external computer, in some embodiments, the processing circuitry including hardware and software can be incorporated in the hub of a neuro attack monitoring device according to the present teachings.

More specifically, referring to FIG. 4D, the NAP device 110 can operate in conjunction with one or more pneumatic cuffs 420 (and/or CO₂ inhalation cup), which can be used to induce the hemodynamic oscillations. Further, the NAP device 110 can be configured to operate in conjunction with other suitable peripheral measurement devices, such as respiratory rate monitors, heart monitors, and/or other patient monitoring devices. For example, a pneumatic cuff can be wrapped around the patient's upper arm and a heart monitor can be positioned at a suitable place on the patient's chest. The peripheral measurement devices can be configured such that they are activated after a predetermined time period has passed following the operation of data acquisition by the NAP device 110. For example, in one embodiment, after collection of baseline data using the NAP device 110 for a predetermined period of time (e.g., 5-10 minutes), the pneumatic cuff, wrapped around the patient's arm or thigh can be inflated to a desired pressure above the normal systolic blood pressure (e.g., 180-200 mmHg), typically to 200 mm of Hg. The cuff can remain inflated for a first predetermined time period (e.g., 2-3 minutes) and then suddenly released. Given that the pneumatic cuff is inflated to above the normal systolic blood pressure, the inflation and sudden release of the cuff can induce a systemic drop (e.g., 10-25 mmHg) in Mean Arterial Pressure (MAP) in patient's body, the recovery to baseline from which can occur within a certain time period, for example, typically 20 seconds. After about 5 minutes of recovery, the cuff (or cuffs) can be cyclically inflated (e.g., to about 200 mmHg) and deflated at five frequencies in the range of 0.03-0.12 Hz for a time period of 2-3 minutes per each frequency (12 minutes total). After 5 minutes of recovery, the data collection can follow. The protocol can last less than about 30 minutes and can provide the required NIRS data. In some cases, during this process, the subject's vital signs can be monitored.

As noted above, in some embodiments, a CO₂ inhalation cup can be used to induce the hemodynamic oscillations through a clinical process called metabolic acidosis in the brain region and collect the NIRS data. The inhaled CO₂ can cause perturbations in the blood flow of the patient and the patient's brain (e.g., both Cerebral Blood Flow and Cerebral Blood Volume). The NAP device can synchronize the acquisition and collection of data to the ingestion of CO₂ by the patient, creating similar temporal or time-varying perturbations as the cuff. The peripheral measurement devices can also be used to monitor the respiratory rate of the subject to ensure that the level of CO₂ (which in some embodiments is typically limited to <than 8%) in the patient's body remains within an acceptable range throughout the entire procedure.

In some embodiments, the collected data can be filtered, e.g., using a low-pass filter, moving average filter, box-car filter and/or a combination of filter such as described above and the filtered temporal data can be converted into the frequency domain using a standard Fourier transform, and the frequency domain data can be analyzed using Coherent Hemodynamic Spectroscopy (CHS). For example, processing of the data can further include converting phasors of physiological observables to hemoglobin concentration phasors, such as brain concentrations of oxygen hemoglobin, deoxy-hemoglobin, and total hemoglobin. Specifically, the NAP device can take advantage of a modified Beer-Lambert law by measuring amplitude decay of continuous wave (CW) IR incident light. Light intensity changes collected at the detector location can be translated into chromophore concentration changes, namely deoxy- and oxy-hemoglobin concentration changes.

For example, in some embodiments, a CHS analysis method disclosed in U.S. Published Application No. 2015/0366514 entitled “Coherent Hemodynamics Spectroscopy and Model Based Characterization of Physiological Systems,” which is herein incorporated by reference in its entirety, can be employed. In the frequency domain, the collected data can be fit into a hemodynamic perturbation model to extract physiological parameters such as, blood transit times, blood volumes in arterial, capillary, and venous compartments, autoregulation cutoff frequency, concentrations of oxy- and deoxy-hemoglobin from the collected data.

An advantage of a system according to the present teachings is that the three-way combination of a NAP device according to the present teachings with the NIRS and CHS methods allows direct measurement of parameters that can identify ischemia of the brain and particularly Transient Ischemic Attack (TIA) or events. Cerebral hemodynamics are commonly assessed by measuring the CBFv, an indirect measurement of Cerebrovascular Reactivity (CVR). In case of ischemic events and impaired hemodynamics, both CBFv and CVR are severely reduced. Research has shown a strong correlation between impaired CBFv and CBV and risk of recurrent stroke or TIA, especially in patient population with carotid artery stenosis and occlusion.

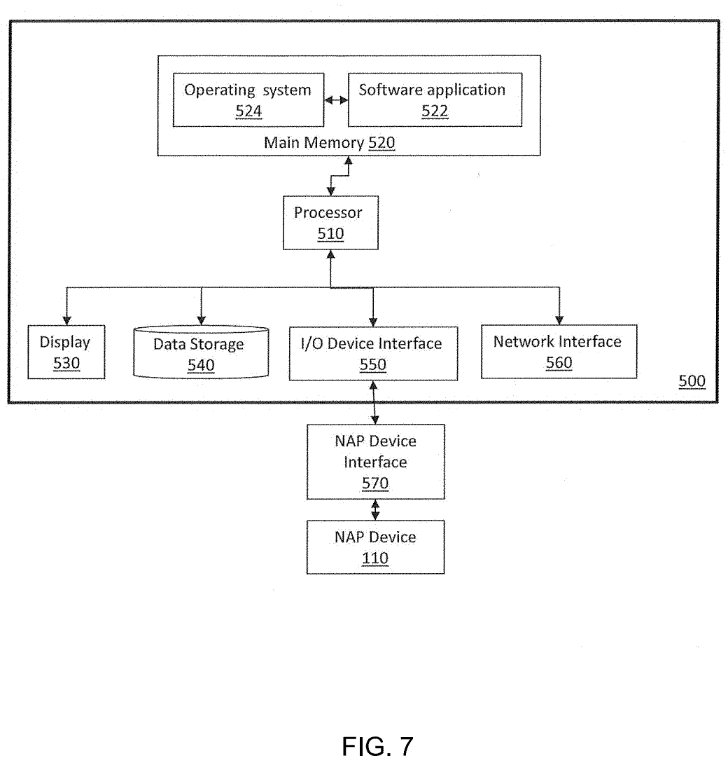

With reference to FIG. 1 as well as FIG. 7, the data collected by the NAP device 110 can be forwarded to a processor or a database (e.g., in the hub 10), for example the processor of a computing device 500, for analysis accordance with the present teachings and/or storage. Alternatively or additionally, the data can be stored in the hub 10. The data can be forwarded from the NAP device 110 through any suitable means known in the art. By way of example, the data can be forwarded from the NAP device using either or a combination of Wifi, Bluetooth, Zigbee and/or NFC technology. In one such embodiment, the NAP device 110 can comprise or be coupled with a Bluetooth chip 122 that provides the NAP device 110 with the ability to communicate with other Bluetooth enabled devices and/or cloud servers. The processor, database, and/or the computing device 500 can also comprise or be coupled with any suitable technology needed to receive the data acquired by the NAP device 110. For example, as described in further details below, the computing device 500 can be coupled with an input/output interface 550 (e.g., Bluetooth chip) that provides the computing device 500 with the ability to communicate with the NAP device 110 (e.g., other Bluetooth enabled devices).

The processor, database, and/or the computing device 500 can comprise any suitable software/hardware available in the art. For example, as shown in FIG. 7, which illustrates a high-level block diagram of the components that can be used in the computing device system 500 used for analyzing the data obtained by the NAP device 110 according to some embodiments disclosed herein. The system 500 can be implemented using suitable techniques informed by the present teachings. Further, the system 500 need not be directly coupled to the NAP device 110 and can be partially or completely independent of the NAP device 110 and connect to the NAP device 110 using any suitable means available in the art. In some embodiments, the system 500 can be implemented in the hub 10 of the NAP device 110.

The system 500 can be a mobile device such as a simple tablet or a phone and/or comprise a processor 510 that carries out some of the functions described herein, such as Neuro Monitoring System 100 data analysis, interpretation, and reporting. Generally these functions disclosed herein can be carried out and implemented by any suitable computer system and/or in digital circuitry or computer hardware. The processor 510 can implement the various functions and methods described herein. For example, the processor 510 can implement application software and procedures that obtain, record, analyze, and/or report the data collected by the NAP device 110. The processor 510 can be connected to the main memory 520. The processor 510 and the main memory 520 can be included in or supplemented by special purpose logic circuitry.

The processor 510 can include a central processing unit (CPU, not shown) that includes processing circuitry configured to manipulate instructions received from the main memory 520 and execute various instructions. For example, the processor 510 can be a general and/or special purpose microprocessor, microcontroller and/or system-on-chip design and any one or more processors of any kind of digital computer. Generally, the processor 510 can be configured to receive instructions and data from the main memory 520 (e.g., a read-only memory or a random access memory or both) and execute the instructions. The instructions and other data can be stored in the main memory 520.

Further, as shown in FIG. 7, the main memory 520 can include an operating system 524. The main memory 520 and the operating system 524 can be configured to implement various operating system functions. For example, the operating system 524 can be responsible for controlling access to various devices, memory management, and/or implementing various functions of the Neuro Monitoring System 100. The main memory 520 can be any form of non-volatile memory included in machine-readable storage devices suitable for embodying data and computer program instructions. For example, the main memory 520 can be magnetic disk (e.g., internal or removable disks), magneto-optical disks, one or more of a semiconductor memory device (e.g., EPROM or EEPROM), flash memory, CD-ROM, and/or DVD-ROM disks.

The main memory 520 can also hold application software 522. For example, the main memory 520 and application software 522 can include various computer executable instructions, application software, and data structures such as computer executable instructions and data structures that implement various aspects of the embodiments described herein. For example, the application software 522 can include various computer executable instructions, application software, and data structures such as computer executable instructions and data structures that can be used to collect, process, and/or report the data obtained from the Neuro Monitoring System 100.

The main memory 520 can also be connected to a cache unit (not shown) configured to store copies of the data from the most frequently used main memory 520. The program codes that can be used with the embodiments disclosed herein can be implemented and written in any form of programming language, including compiled or interpreted languages, and can be deployed in any form, including as a stand-alone program, an App or as a component, module, subroutine, or other unit suitable for use in a computing environment. A computer program can be configured to be executed on a computer, or on multiple computers, a farm of cloud servers at one site or distributed across multiple sites and interconnected by a communications network, such as the Internet.

The functions performed by the Neuro Monitoring System 100, such as operating the light sources and detectors, controlling collection of data by the detectors, analysis of the data obtained by the detectors, and reporting of the analyzed data can be implemented in digital electronic circuitry or in computer hardware that executes software, firmware, or combinations thereof. The implementation can be as a computer program product, for example a computer program tangibly embodied in a non-transitory machine-readable storage device, for execution by, or to control the operation of, data processing apparatus, for example a computer, a programmable processor, or multiple computers.

Further, as shown in FIG. 7, the processor 510 can also be connected to various interfaces via a system or an input/output (I/O) interface 550 (e.g., Bluetooth, USB connector, audio interface, FireWire, interface for connecting peripheral devices, etc.). The I/O interface 550 can directly or indirectly connect to the NAP device 110. For example, in the embodiment shown in FIG. 7, the I/O interface 550 connects to the NAP device 110 through an interface 570 of the NAP device 110.

Further, the NAP device 110 can use the detectors to collect observation data from the patient and forward the collected data to the processor for processing and/or presenting it to a user (not shown) on the display 530 of the system 500. Any data obtained from collecting and/or analyzing the collected data can further be stored on either physical storage or on the cloud servers on the internet, a data storage 540 of the system 500.

The display 530 can be any suitable display available in the art, for example a Liquid Crystal Display (LCD) or a light emitting diode (LED) display. The display can further be a touch screen display that can receive instructions from a user.

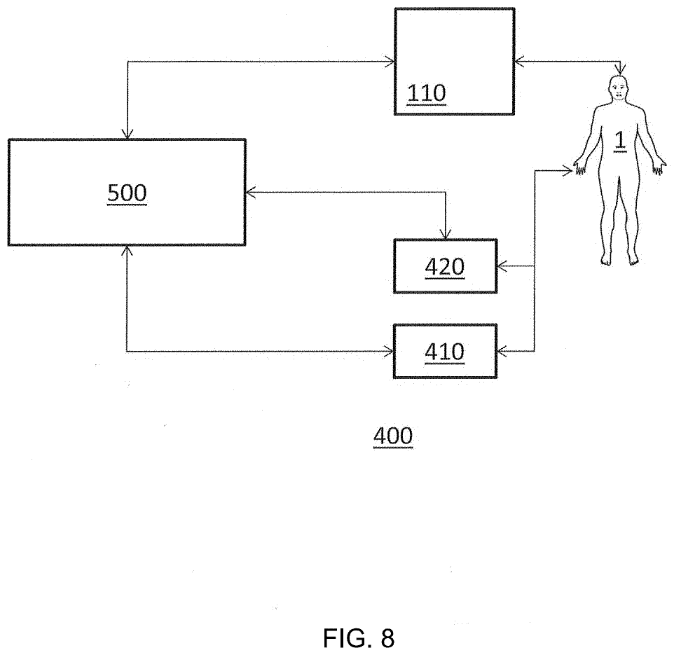

The processor 510 can also control the functions of the NAP device 110 in response to instructions received from the main memory 520 and the software application 522. The software application or an App, 522 can further include software applications that can store and process the data obtained by the NAP device 110. Examples of such processing can include pre-processing, processing, interpreting, and reporting the data. The I/O interface 550 can further be connected to other peripherals, such as the peripheral devices 410, 420 described with reference to FIG. 4D (See also FIG. 8), one or more speakers for acoustic output, a microphone for acoustic input, or any other suitable peripheral device known in the art.

The processor 510 can also be connected a network/cloud interface 560. The communications interface or a farm of cloud servers 560 can provide the NAP device 110 with a connection to a communications network, such as the Internet. Transmission and reception of data, information, and instructions can occur over the communications network. In one embodiment, NAP data can also be processed using a farm of single site or distributed cloud servers and other machine learning algorithms using available and known techniques in the art.

By way of further illustration, FIG. 6 illustrates an example of a high-level block diagram of a neuro attack monitoring system according to some embodiments disclosed herein. As noted above, the NAP device 110 can be used to collect data from a patient 1. The NAP device 110 can be connected to the computing device 500 through any suitable means available in the art (e.g., directly, wirelessly, such as through Wifi/Bluetooth/Zigbee/NFC connection). The computing device 500 can control the operation of the NAP device 110 by sending signals (e.g., control signals). For example, the computing device 500 can send signals to the NAP device 110 that power on or power off the device and/or signal to the device to commence the data acquisition process. The computing device 500 can also control the operation of the NAP device 110 by setting any variable or parameter involved in the data acquisition and collection process, such as acquisition cycles, acquisition frequencies, acquisition time periods, signal intensities, laser intensities, and/or modulation frequencies of the NAP device 110 and components incorporated in the NAP device (e.g., the light sources and detectors). In addition to providing the NAP device 110 with control signals, the computing device 500 can also receive information (e.g., acquired data, control signals, etc.) from the NAP device 110.

The computing device 500 can also control the functions of any peripheral devices 410, 420 (e.g., inflation cuff, CO₂, heart rate monitor, respiratory rate monitor, etc.) that are used in conjunction with the NAP device 110. For example, the computing device 500 can provide the peripheral devices 410, 420 with any control signals necessary to initiate, end, or control the operation of these devices. The computing device 500 can also receive control signals and data, including any data collected by the peripheral devices 410, 420 from these devices 410, 420.

The computing device can also provide the NAP device 110 and the components included in the NAP device, and the peripheral devices 410, 420 with power. Generally, the computing device 500, the NAP device 110 and the components included in the NAP device, and the peripheral devices 410, 420 can be operated and powered using any suitable available technique. For example, at least one of these components can be a battery-operated component. The battery can be any battery known in the art, for example a reusable and/or rechargeable battery. In some embodiments, the computing device 500 can include the components or functionality necessary to recharge or cause the recharging of batteries included in the NAP device 110 and/or the peripheral devices 410, 420. In some embodiments, the NAP device can be powered using a lithium-ion battery, having for example at least four hours of continuous operation capacity on a single charge.

As noted above, in some embodiments, the data collected by the detectors can be transmitted to the hub 10 or forwarded to the computing device and or cloud servers for storage and further analysis. The converted signals can be time-stamped signals. The acquisition software can add a date and time stamp to the data collected. In addition to the acquired data, the computing device can receive information regarding the peripheral devices and their operational characteristics, such as their operating frequency. For example, the computing device can send control signals to the peripheral devices that control the frequency of blood flow perturbation by regulating the timing of the operation of the peripheral device (e.g., the timing of the operation of an inflation cup or an upper arm cuff). As noted above, one such peripheral device is an inflation cuff that can be used to increase the systolic pressure. Such an inflation cuff can be coupled to the arm or more preferably to the thigh of a subject. The computing device can also collect data obtained by the peripheral devices from these devices.

The computing device can use the collected information to detect ischemic events, such as stroke, particularly TIA in the brain, detect traumatic injury in the brain, and/or determine oximetry (oxygen saturation) of brain tissue (box 760 in FIG. 9). In this embodiment, the NAP device can implement CHS technique in time domain by measuring a hemodynamic transient signal. The hemodynamic oscillations so derived can be used in the quantification of the physiological parameters (blood transit times, blood volumes in arterial, capillary, and venous compartments, autoregulation cutoff frequency, etc.). As such, the present disclosure provides a three-way combination of the NAP device with the NIRS and CHS technique to directly measure parameters that can identify ischemia of the brain and particularly Transient Ischemic Attack (TIA) or events.

The following Example is provided for further elucidation of various aspects of the present teachings and is not intended to be limiting of the scope of the invention.

### Example

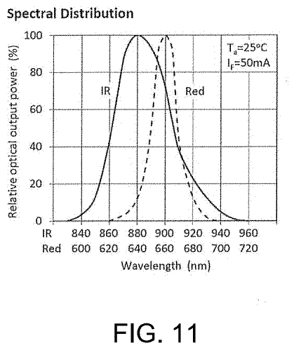

A physical hardware model was created in order to evaluate the performance of an example of a system according to the present disclosure. The model approximated the full signal path from the infrared (IR) light sources to the receivers through a patient's body in order to derive the signal required as input into the CHS algorithm. A near-infrared spectrum produced by commercially available IR diode emitters generating radiation at wavelengths of 650 and 850 nm was employed. A typical output spectrum is shown in FIG. 11. The light from the IR emitters was then attenuated by a model of the tissue employed in this example.

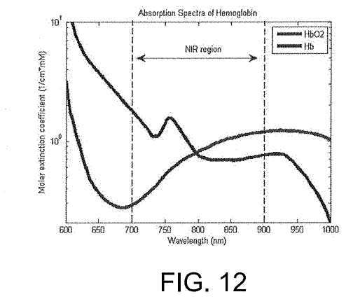

The distance traversed by the light between the emitter and receiver was assumed to be nominal 2.5 cm. The hemoglobin extinction coefficients shown in FIG. 12 were used to attenuate the light in the tissue using modified Beer-Lambert Law:

$$OD \approx {OD}_{0} + ( \frac{\partial {OD}_{0}}{\partial {μ}_{a}} ) {Δμ}_{a} + ( \frac{\partial {OD}_{0}}{{Δμ}_{s}^{′}} ) {Δμ}_{s}^{′}$$

where OD is the optical density, OD0 is the baseline optical density, Δμₐ and Δμ′ₛ are differential changes in absorption and scattering coefficients.

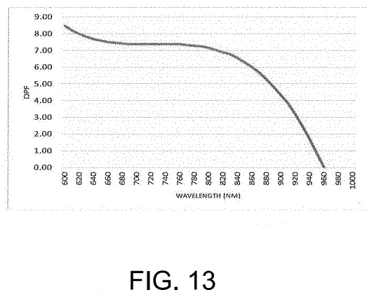

The above equation can be simplified to the following relation:

$$Δ OD = OD - {OD}_{0} = - log ( \frac{I ( t )}{{I}_{s}} ) \approx 〈 L 〉 {Δμ}_{a} ( t ) + ( \frac{{{μ}_{a}}_{0}}{{μ}_{{s}_{0}^{′}}} ) 〈 L 〉 {Δμ}_{s}^{′} ( t ) \approx 〈 L 〉 {Δμ}_{a} ( t )$$

where [symbol] L [symbol] is the product of the source-to-receiver distance multiplied by a differential path factor (DPF), which accounts for the scattering of the light inside the tissue. The DPF is a function of the light wavelength and other biological factors, such as patient's age and tissue composition. A polynomial approximation introduced by Scholkmann and Wolf, described in an article entitled “General equation for the differential pathlength factor of the frontal human head depending on wavelength and age.”, J. Biomed Opt, 2013, October; 18(10):105004.
which is herein incorporated by reference in its entirety, was used to calculate the DPF, shown as a function of wavelength for an adult brain in FIG. 13. The DPF is a function of the light wavelength and other biological factors, such as patient's age and tissue composition. Standard absolute and relative hemoglobin concentrations were used for the calculations.

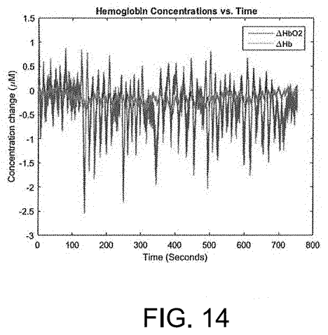

Lastly, the attenuated spectra were folded with the light conversion efficiency of a standard off-the-shelf IR receiver. The absorption changes resulting from the simulated light signals were used in the Beer's law to measure changes in Hb and HbO₂ concentrations. The results are shown in FIG. 14. FIG. 14 shows the data with the signal values of the de-oxygenated hemoglobin (Hb) concentration and oxygenated hemoglobin (HbO2) concentration as modeled by the NAP physical model. The concentration changes as a function of time were well reproduced by the physical model. The slight differences could be explained by specific aspects of the measurement, such as optical coupling or a less accurate choice of the DPF.

While the invention has been particularly shown and described with reference to specific illustrative embodiments, it should be understood that various changes in form and detail may be made without departing from the spirit and scope of the invention. Further, it is to be appreciated that various alterations, modifications, and improvements will readily occur to those skilled in the art. Such alterations, modifications, and improvements are intended to form a part of this disclosure, and are intended to be within the spirit and scope of this disclosure. While some examples presented herein involve specific combinations of functions or structural elements, it should be understood that those functions and elements may be combined in other ways according to the present disclosure to accomplish the same or different objectives. In particular, acts, elements, and features discussed in connection with one embodiment are not intended to be excluded from similar or other roles in other embodiments. Additionally, elements and components described herein may be further divided into additional components or joined together to form fewer components for performing the same functions.

What is claimed is:

1. A device comprising:
a central hub configured for placement on a patient's head; and
a plurality of probes connected to the central hub and configured for placement on the patient's head over a specific portion of the patient's brain, at least one of said probes comprising one or more pairs of light emitting sources and at least one light detector;
wherein the light emitting sources are configured to inject light into the patient's head, at two or more different wavelengths, over a predetermined period of time.

2. The device of claim 1, wherein at least one of the probes comprises two portions hingedly coupled to one another via a swivel hinge.

3. The device of claim 2, wherein said swivel hinge is configured to allow adjusting an angle between said probes in a range of about 15° to about 30° degrees.

4. The device of claim 3, wherein each of said hingedly-coupled probe portions comprises a detector.

5. The device of claim 2, wherein at least one of said hingedly-coupled probe portions comprises at least one emitter.

6. The device of claim 1, wherein each probe comprises a spoke extending from said central hub to a pod.

7. The device of claim 1, wherein at least the pod associated with said at least one probe is configured to house said one or more pairs of light emitting sources and at least one light detector.

8. The device of claim 1, wherein said at least one detector comprises a photodiode.

9. The device of claim 1, wherein at least one of said pairs of emitters generates radiation at a wavelength in a range of about 650 to about 710 nm and another one of said pairs of emitters generates radiation at a wavelength in a range of about 800 nm to about 830 nm.

10. The device of claim 1, further comprising a switching module for switching the source wavelengths between different wavelengths in the range of 690 nm-830 nm.

11. The device of claim 10, wherein said switching module is configured to pulse a single emitter via a time delay circuitry in the hub to generate different radiation wavelengths.

12. The device of claim 1, wherein said at least one detector is configured to receive at least a portion of the radiation emitted by at least one of the light emitting sources after passage thereof through a portion of the patient's brain and to generate at least one detection signal

13. The device of claim 11, further comprising circuitry implementing a pre-processing algorithm for application to said at least one detection signal.

14. The device of claim 11, wherein said pre-processing algorithm comprises a combination of higher-order polynomial interpolation and a low-pass filter.

15. The device of claim 11, further a computing device for operating on said detection signal for determining an ischemic event.

16. The device of claim 14, wherein said ischemic event comprises a full ischemic stroke.

17. The device of claim 14, wherein said ischemic event comprises a transient ischemic event.

18. The device of claim 15, wherein said computing device is configured to operate on said at least one detection signal to determine CBFv and to apply a threshold to said CBFv to distinguish a baseline from an ischemic event.

19. The device of claim 13, wherein said computing device is housed within said hub.

20. The device of claim 13, wherein said computing device is external to said hub.

21. The device of claim 1, further comprising a controller for controlling operation of said light emitting sources and said light detector.

22. The device of claim 20, wherein said controller is disposed in said hub.

23. The device of claim 20, wherein said hub interfaces with external devices or cloud servers in a single site or distributed multiple sites for data storage, data processing and data analysis.

24. A system for neuro attack monitoring, comprising:
a monitoring device, comprising:
a central hub configured for placement on a patient's head; and
a plurality of probes connected to the central hub and configured for placement on the patient's head over a specific portion of the patient's brain, at least one of said probes comprising one or more pairs of light emitting sources and at least one light detector;
wherein the light emitting sources are configured to inject light into the patient's head, at two or more different wavelengths, over a predetermined period of time, and a computing device for receiving data from said at least one light detector and operating on said data to determine onset of an ischemic event.

25. The system of claim 24, wherein said computing device employs coherent hemodynamic spectroscopy method to analyze the data.
